(19) United States
(12) Patent Application Publication
(10) Pub. No.: US 2026/0294539 A1
(43) Pub. Date: Oct. 1, 2026

(54) SURGICAL LASER SYSTEM CONTROL BASED ON INTERPOLATED FIBER DAMAGE FOR LASER LITHOTRIPSY

(71) Applicant: Lumenis Ltd., Yokneam Ilit (IL)

(72) Inventors: Amir Finkel, ZICHRON YAAKOV (IL); Saman Naser, Tira (IL)

(73) Assignee: Lumenis Ltd., Yokneam Ilit (IL)

(21) Appl. No.: 19/635,876

(22) Filed: Apr. 1, 2026

Related U.S. Application Data
(60) Provisional application No. 63/781,572, filed on Apr. 1, 2025.

(51) Int. Cl. A61B 18/26; A61B 17/00; A61B 18/00

(52) U.S. Cl. CPC A61B 18/26; A61B 2017/00115; A61B 2018/00577; A61B 2018/00702

(57) ABSTRACT

The present disclosure provides a surgical laser system configured to interpolate damage to an optical fiber in which laser energy is transmitted to ablate a target. The surgical laser system is configured to measure, with one or more signal detector, intensity of parasitic reflections from the proximal and distal facets of the optical fiber. Further, the surgical laser system is configured to interpolate damage to the optical fiber based on the measured intensity and to dynamically adjust operation parameters and/or display alerts to a user based on the interpolated damage.

### PRIORITY

This application claims the benefit of priority under 35 USC § 119 to U.S. Provisional Patent Application Ser. No. 63/781,572, filed Apr. 1, 2025, which is incorporated by reference herein in its entirety and for all purposes.

### TECHNICAL FIELD

The present disclosure generally relates to surgical laser systems used in medical or therapeutic procedures. Particularly, but not exclusively, the present disclosure relates to systems and methods to dynamically control the power delivered by the surgical laser system based on damage to an optical fiber during a procedure.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, some stones are required to be fragmented into smaller pieces. A technology known as laser lithotripsy may be used for such fragmenting processes.

In an example lithotripsy procedure, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it.

These treatments require high amounts of accuracy to ensure that the laser is aimed at the right target (stone, tissue, etc.), to achieve the clinical objectives (e.g., stone fragmentation, stone dusting, tissue coagulation, etc.) It is to be appreciated that the treatment laser power delivered from the system depends on the transmission efficiency of the optical fiber.

Thus, there is a need for improved surgical laser systems configured to adjust the power delivered from the system based on the transmission efficiency of the optical fiber.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter. The present disclosure applies to surgical laser systems configured to deliver laser energy to a target via an optical fiber. The surgical laser system includes light detectors arranged to detect optical signals and provides to dynamically control power delivered by the surgical laser system based on damage to the optical fiber. In general, the present disclosure provides to estimate optical fiber damage based on optical signals reflected from the distal and proximal ends of the optical fiber.

Typically, for each lithotripsy procedure, a new optical fiber is user. While the optical fiber is new (e.g., at the beginning of the procedure), the power delivered by the optical fiber is substantially similar to the power specified in the configuration of the surgical laser system (e.g., as set by a user, or the like). However, during the procedure, the proximal and distal ends of the optical fiber are damaged. For example, the proximal and distal ends of the optical fiber are damaged by the laser energy. Further, the distal end of the optical fiber may be damaged by impact from stone fragments, or the like. As such, the overall transmission efficiency of the optical fiber degrades, and the power delivered during the procedure drops. As the optical fiber is continually damaged during the procedure, the transmission efficiency and thus the delivered power may continually degrade. As a result, ablation rates (e.g., stone fragmentation rate, stone dusting rate, etc.) may deteriorate and the overall treatment efficiency may be reduced. Accordingly, the present disclosure can be applied to dynamically adjust output power from the surgical laser system to maintain requested power or the power specified in the configuration of the system despite degradation to the optical fiber.

The disclosure can be implemented as a method for a surgical laser system. The method can comprise generating, via a laser source, an interrogation laser beam; transmitting the interrogation laser beam through an optical fiber coupled to the surgical laser system; measuring, via at least one signal detector, an intensity of a parasitic reflection, wherein the parasitic reflection comprises at least proximal facet reflections and distal facet reflections; determining whether the intensity of the parasitic reflection is less than a baseline intensity threshold; and generating a control signal for the surgical laser system based on a determination that the intensity of the parasitic reflection is less than the baseline intensity threshold.

In further embodiments, the method can comprise deriving a change in the intensity of the parasitic reflection over a time period; and determining whether the change in the intensity is greater than an intensity change threshold, wherein generating the control signal for the surgical laser system is further based on a determination that the change in the intensity is greater than the intensity change threshold.

In further embodiments of the method, the control signal comprises an indication for a therapeutic laser source and is configured to cause the therapeutic laser source to increase, by a specified amount, a pulse frequency, pulse duration, and/or pulse energy of a therapeutic laser beam generated by the therapeutic laser source.

In further embodiments of the method, the control signal comprises an indication for a display and is configured to cause the display to display a suggestion to increase a pulse frequency, pulse duration, and/or pulse energy of a therapeutic laser beam generated by a therapeutic laser source.

In further embodiments, the method can comprise generating the therapeutic laser beam via the therapeutic laser source; illuminating, via the optical fiber, a target with the therapeutic laser beam; and deriving the specified amount of the pulse frequency increase, the pulse duration increase, and/or the pulse energy increase based on the intensity of the parasitic reflection.

In further embodiments of the method, the control signal comprises an indication for a therapeutic laser source and is configured to cause the therapeutic laser source to stop generating a therapeutic laser beam.

In further embodiments of the method, the control signal comprises an indication for a display and is configured to cause the display to display an indication that an efficiency of the energy delivery via the optical fiber is reduced.

In further embodiments of the method, the control signal comprises an indication for a display and is configured to cause the display to display an indication to cleave and/or replace the optical fiber.

In further embodiments of the method, the at least one signal detector comprises a first signal detector and a second signal detector and wherein measuring the intensity of the parasitic reflection via the at least one signal detector comprises measuring an intensity of the proximal facet reflections via the first signal detector; and measuring an intensity of the distal facet reflections via the second signal detector.

In further embodiments, the method can comprise measuring, via the at least one signal detector, an intensity of a dark current; determining whether the intensity of the dark current is greater than a dark current baseline threshold; and wherein generating the control signal for the surgical laser system is further based on a determination that the intensity of the dark current is greater than the dark current baseline threshold.

In further embodiments, the method can comprise deriving normalized intensity based on the measured intensity of the parasitic reflection and the intensity of the dark current, wherein determining whether the intensity of the parasitic reflection is less than the baseline intensity threshold comprises determining whether the normalized intensity is less than the baseline intensity threshold.

In further embodiments, the method can comprise illuminating, via the optical fiber, a target with the therapeutic laser beam; measuring, via the at least one signal detector, the intensity of the parasitic reflection and an intensity of a target reflection; estimating a distance between a distal end of the optical fiber and the target based on the intensity of the parasitic reflection and the intensity of the target reflection.

In further embodiments of the method, the interrogation laser beam comprises a plurality of wavelength components and wherein measuring, via the at least one signal detector, the intensity of the parasitic reflection comprises measuring an intensity at each of the plurality of wavelength components.

Some embodiments of the disclosure can be implemented as a surgical laser system. The surgical laser system can comprise a therapeutic laser source configured to output a therapeutic laser beam; at least one laser source configured to output an interrogation laser beam; at least one signal detector configured to measure an intensity of a parasitic reflection at each of the plurality of wavelength components; a processor; and a memory comprising instructions, which when executed by the processor cause the surgical laser system to generate, via the at least one laser source, the interrogation laser beam, transmit the interrogation laser beam through an optical fiber coupled to the surgical laser system, measure, via the at least one signal detector, an intensity of a parasitic reflection, wherein the parasitic reflection comprises at least proximal facet reflections and distal facet reflections, determine whether the intensity of the parasitic reflection is less than a baseline intensity threshold, and generate a control signal for the surgical laser system based on a determination that the intensity of the parasitic reflection is less than the baseline intensity threshold.

In further embodiments of the surgical laser system, the instructions when executed by the processor further cause the surgical laser system to derive a change in the intensity of the parasitic reflection over a time period; and determine whether the change in the intensity is greater than an intensity change threshold, wherein the control signal for the surgical laser system is generated based on a determination that the change in the intensity is greater than the intensity change threshold.

In further embodiments of the surgical laser system, the control signal comprises an indication for the therapeutic laser source and is configured to cause the therapeutic laser source to increase, by a specified amount, a pulse frequency, pulse duration, and/or pulse energy of the therapeutic laser beam generated by the therapeutic laser source.

In further embodiments of the surgical laser system, the control signal comprises an indication for a display and is configured to cause the display to display a suggestion to increase a pulse frequency, pulse duration, and/or pulse energy of the therapeutic laser beam generated by the therapeutic laser source.

In further embodiments of the surgical laser system, the instructions when executed by the processor further cause the surgical laser system to generate the therapeutic laser beam via the therapeutic laser source; illuminate, via the optical fiber, a target with the therapeutic laser beam; and derive the specified amount of the pulse frequency increase, the pulse duration increase, and/or the pulse energy increase based on the intensity of the parasitic reflection.

In further embodiments of the surgical laser system, the control signal comprises an indication for the therapeutic laser source and is configured to cause the therapeutic laser source to stop generating the therapeutic laser beam.

In further embodiments of the surgical laser system, the control signal comprises an indication for a display and is configured to cause the display to display an indication that an efficiency of the energy delivery via the optical fiber is reduced.

In further embodiments of the surgical laser system, the control signal comprises an indication for a display and is configured to cause the display to display an indication to cleave and/or replace the optical fiber.

In further embodiments of the surgical laser system, the at least one signal detector comprises a first signal detector and a second signal detector and wherein the instructions when executed by the processor further cause the surgical laser system to measure an intensity of the proximal facet reflections via the first signal detector; and measure an intensity of the distal facet reflections via the second signal detector.

In further embodiments of the surgical laser system, the instructions when executed by the processor further cause the surgical laser system to measure, via the at least one signal detector, an intensity of a dark current; determine whether the intensity of the dark current is greater than a dark current baseline threshold; and wherein the control signal for the surgical laser system is generated based on a determination that the intensity of the dark current is greater than the dark current baseline threshold.

In further embodiments of the surgical laser system, the instructions when executed by the processor further cause the surgical laser system to derive normalized intensity based on the measured intensity of the parasitic reflection and the intensity of the dark current; and determining whether the normalized intensity is less than the baseline intensity threshold to determine whether the intensity of the parasitic reflection is less than the baseline intensity threshold.

In further embodiments of the surgical laser system, the instructions when executed by the processor further cause the surgical laser system to illuminate, via the optical fiber, a target with the therapeutic laser beam; measure, via the at least one signal detector, the intensity of the parasitic reflection and an intensity of a target reflection; estimate a distance between a distal end of the optical fiber and the target based on the intensity of the parasitic reflection and the intensity of the target reflection.

In further embodiments of the surgical laser system, the interrogation laser beam comprises a plurality of wavelength components and wherein the instructions when executed by the processor further cause the surgical laser system to measure, via the at least one signal detector, an intensity at each of the plurality of wavelength components to measure the intensity of the parasitic reflection.

Some embodiments of the disclosure can be implemented as at least one non-transitory machine-readable storage device comprising instructions, which when executed by a processor of a surgical laser system cause the surgical laser system to generate, via at least one laser source, an interrogation laser beam; transmit the interrogation laser beam through an optical fiber coupled to the surgical laser system; measure, via at least one signal detector, an intensity of a parasitic reflection, wherein the parasitic reflection comprises at least proximal facet reflections and distal facet reflections; determine whether the intensity of the parasitic reflection is less than a baseline intensity threshold; and generate a control signal for the surgical laser system based on a determination that the intensity of the parasitic reflection is less than the baseline intensity threshold.

In further embodiments of the at least one non-transitory machine-readable storage device, the instructions when executed by the processor further cause the surgical laser system to derive a change in the intensity of the parasitic reflection over a time period; and determine whether the change in the intensity is greater than an intensity change threshold, wherein the control signal for the surgical laser system is generated based on a determination that the change in the intensity is greater than the intensity change threshold.

In further embodiments of the at least one non-transitory machine-readable storage device, the control signal comprises an indication for the therapeutic laser source and is configured to cause the therapeutic laser source to increase, by a specified amount, a pulse frequency, pulse duration, and/or pulse energy of the therapeutic laser beam generated by the therapeutic laser source.

In further embodiments of the at least one non-transitory machine-readable storage device, the control signal comprises an indication for a display and is configured to cause the display to display a suggestion to increase a pulse frequency, pulse duration, and/or pulse energy of the therapeutic laser beam generated by the therapeutic laser source.

In further embodiments of the at least one non-transitory machine-readable storage device, the instructions when executed by the processor further cause the surgical laser system to generate the therapeutic laser beam via the therapeutic laser source; illuminate, via the optical fiber, a target with the therapeutic laser beam; and derive the specified amount of the pulse frequency increase, the pulse duration increase, and/or the pulse energy increase based on the intensity of the parasitic reflection.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates a surgical laser system in accordance with embodiments of the disclosure.

FIG. 1B illustrates a portion of the surgical laser system of FIG. 1A in greater detail.

FIG. 2 illustrates an aspect of the subject matter in accordance with one embodiment.

FIG. 3 illustrates an aspect of the subject matter in accordance with one embodiment.

FIG. 4 illustrates a first interrogation sub-system in accordance with embodiments of the disclosure.

FIG. 5 illustrates a method for controlling a laser source to stabilize signals at a detector in accordance with embodiments of the disclosure.

FIG. 6 illustrates a method for controlling a laser source to stabilize signals at a detector in accordance with embodiments of the disclosure.

FIG. 7 illustrates a method for controlling a laser source to stabilize signals at a detector in accordance with embodiments of the disclosure.

FIG. 8 illustrates a method for controlling a laser source to stabilize signals at a detector in accordance with embodiments of the disclosure.

FIG. 9 illustrates a method for controlling a laser source to stabilize signals at a detector in accordance with embodiments of the disclosure.

FIG. 10 illustrates a computer-readable storage medium in accordance with embodiment of the disclosure.

FIG. 11 illustrates a specially programmed computing system in accordance with embodiments of the disclosure.

### DETAILED DESCRIPTION

The present disclosure applies to surgical laser systems with light detectors arranged to detect optical signals. An illustrative surgical laser system can be provided with both therapeutic and interrogative laser sources and can be configured to determine a transmission efficiency of an optical fiber coupled to the surgical laser system and to dynamically adjust output power based on the determined transmission efficiency.

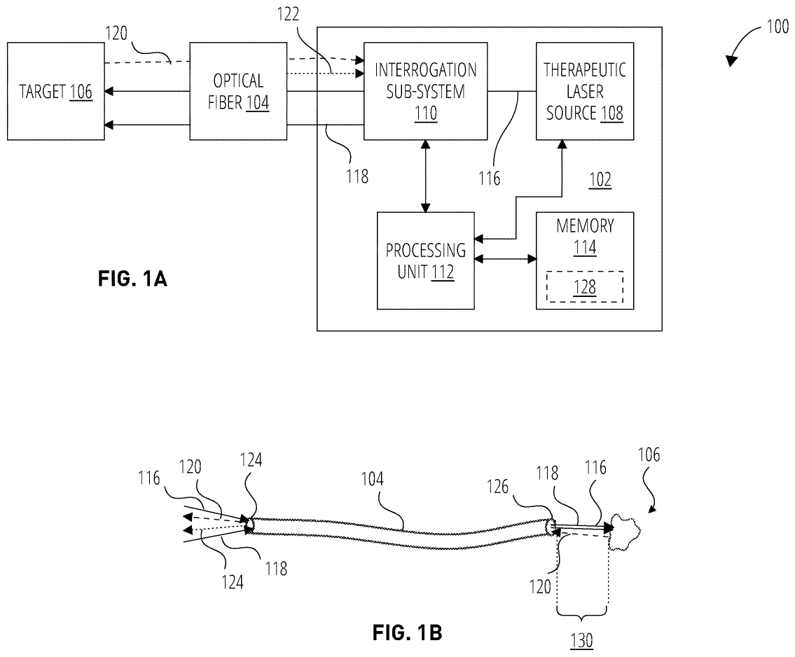

FIG. 1A show an exemplary surgical laser system 100 for determining a transmission efficiency of an optical fiber. It is noted that the surgical laser system 100 can further be configured to measure a distance between a distal end of the optical fiber and a target. The surgical laser system 100 comprises a surgical laser console 102 and an optical fiber 104. To aid the discussion, the surgical laser system 100 is depicted treating a target 106. In an exemplary lithotripsy procedure, the target 106 can be a stone. However, it will be appreciated that the surgical laser console 102 and optical fiber 104 could be used to treat other types of targets (e.g., tissue, a tumor, a cyst, or the like). With illustrative examples, the target can be disposed in a liquid environment within a subject (e.g., a human, an animal, or the like).

The surgical laser console 102 includes a therapeutic laser source 108, an interrogation sub-system 110, a processing unit 112, and a memory 114. During operation, the therapeutic laser source 108 is configured to generate therapeutic laser pulses 116 while interrogation sub-system 110 is configured to generate interrogation laser pulses 118.

In general, the therapeutic laser source 108 can be any of a variety of laser light sources (e.g., solid-state lasers, gas lasers, diode lasers, and fiber lasers). As a specific example, the therapeutic laser source 108 can be a Holmium (Ho) laser, such as a Ho:YAG laser. In yet another example, the therapeutic laser source 108 can be a Thulium (Tm) fiber laser.

An example of the interrogation sub-system 110 is provided in greater detail below. However, in general the interrogation sub-system 110 is configured to generate interrogation laser pulses 118, which comprise multiple (e.g., two, three, four, etc.) laser beams each having different wavelengths.

The therapeutic laser pulses 116 and the interrogation laser pulses 118 are transmitted towards the target 106 via the optical fiber 104. Target reflected laser pulses 120 are light reflected from the target responsive to the therapeutic laser pulses 116 and/or the interrogation laser pulses 118 being incident on the target as well as light reflected from other features, referred to herein as parasitic reflections 122. This is depicted in greater detail in FIG. 1B, which depicts the optical fiber 104. The optical fiber 104 includes both a proximal end 124 and a distal end 126. The proximal end 124 is the end of the optical fiber 104 coupled to the surgical laser console 102 and through which therapeutic laser pulses 116 and interrogation laser pulses 118 enter while the distal end 126 is the end of the optical fiber 104 through which the therapeutic laser pulses 116 and interrogation laser pulses 118 are emitted. During a procedure, the physician can manipulate the distal end 126 of the optical fiber 104 to “point” the optical fiber 104 at the target 106 and to place the distal end 126 within a desired distance to the target 106.

As outlined above, the proximal end 124 and the distal end 126 of the optical fiber 104 are damaged during the procedure. For example, the therapeutic laser pulses 116 will damage the proximal end 124 and the distal end 126 while fragments from the target 106 may strike the distal end 126 and cause damage to the distal end. As such, the overall transmission efficiency of the optical fiber 104 will degrade. At the beginning of the procedure, when the optical fiber 104 is new and not damaged, the power of the therapeutic laser pulses 116 delivered or emitted from the distal end 126 will substantially match the power of the therapeutic laser pulses 116 generated by the therapeutic laser source 108. However, as the optical fiber 104 is damaged and the transmission efficiency degrades, the power the power of the therapeutic laser pulses 116 delivered or emitted from the distal end 126 will also degrade and will longer substantially match the power of the therapeutic laser pulses 116 generated by the therapeutic laser source 108.

The present disclosure provides to measure or estimate the damage of the optical fiber 104 based on reflections from the proximal end 124 and the distal end 126 of the optical fiber 104. Further, the disclosure provides to dynamically adjust the power of the therapeutic laser pulses 116 generated by the therapeutic laser source 108 such that the power of the therapeutic laser pulses 116 emitted from the distal end 126 of the optical fiber 104 matches the power expected or requested by configuration of the surgical laser system 100.

The processing unit 112 can include circuitry arranged to execute instructions 128 stored in memory 114, which instructions when executed cause the processing unit 112 to send control signals to the interrogation sub-system 110 to measure parasitic reflections 122 (or portions of the parasitic reflections 122) and account for the measured parasitic reflections 122 as part of dynamically controlling the power of the therapeutic laser pulses 116 to account for degradation of the optical fiber 104 during a procedure. With some examples, the processing unit 112 and memory 114 may be provided by a computing device (e.g., a laptop, a desktop, a mobile phone, a tablet, or the like).

It is to be appreciated that the surgical laser console 102 and particularly the therapeutic laser source 108 and interrogation sub-system 110 can include a variety of optical components besides the laser sources described above. For example, therapeutic laser source 108 and interrogation sub-system 110 can include (e.g., in different combinations as may be the case) light sources, polarizers, beam splitters, beam combiners, light detector, wavelength division multiplexers, collimators, circulators, etc.

Further, although not depicted, the surgical laser console 102 could include a display and/or other input and/or output devices configured to provide interaction with the surgical laser console 102 by a user (e.g., physician, or the like).

It is to be appreciated that the target 106 is often in a liquid environment (e.g., urine, water, blood, etc.) For example, during a lithotripsy procedure, the target 106 may be a stone and may be disposed in an environment that includes a mixture of urine, irrigation fluid, and blood. Accordingly, the distal end 126 of the optical fiber 104 will also be disposed in the liquid environment during the procedure. When the therapeutic laser pulses 116 are emitted from the distal end 126 of the optical fiber 104, some of the liquid may be vaporized and form a bubble or bubbles.

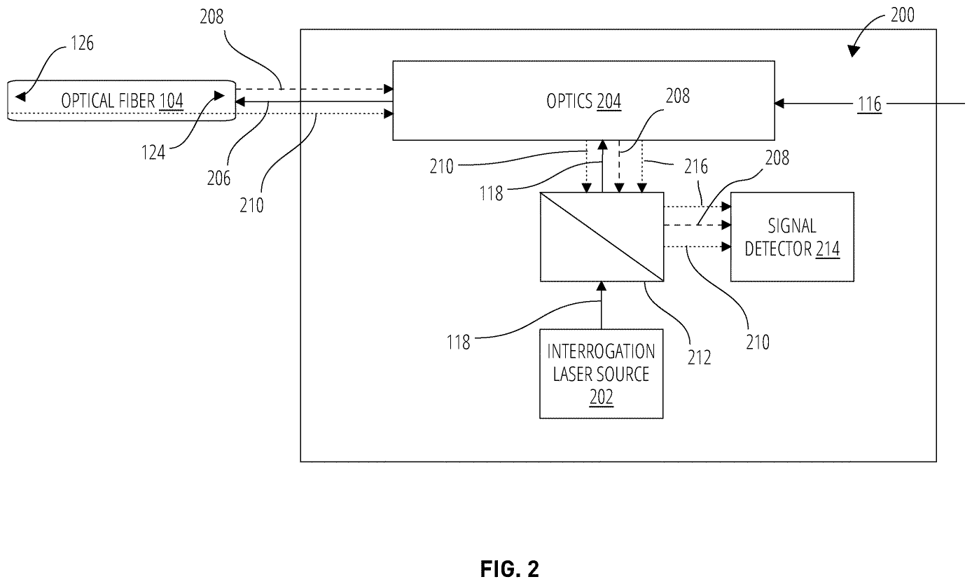

FIG. 2 illustrates an example interrogation sub-system 200, which can be provided in accordance with some embodiments of the present and used to interpolate optical fiber damage. For example, the interrogation sub-system 200 can be provided as the interrogation sub-system 110 of the surgical laser console 102 shown as part of the surgical laser system 100 in FIG. 1A and FIG. 1B.

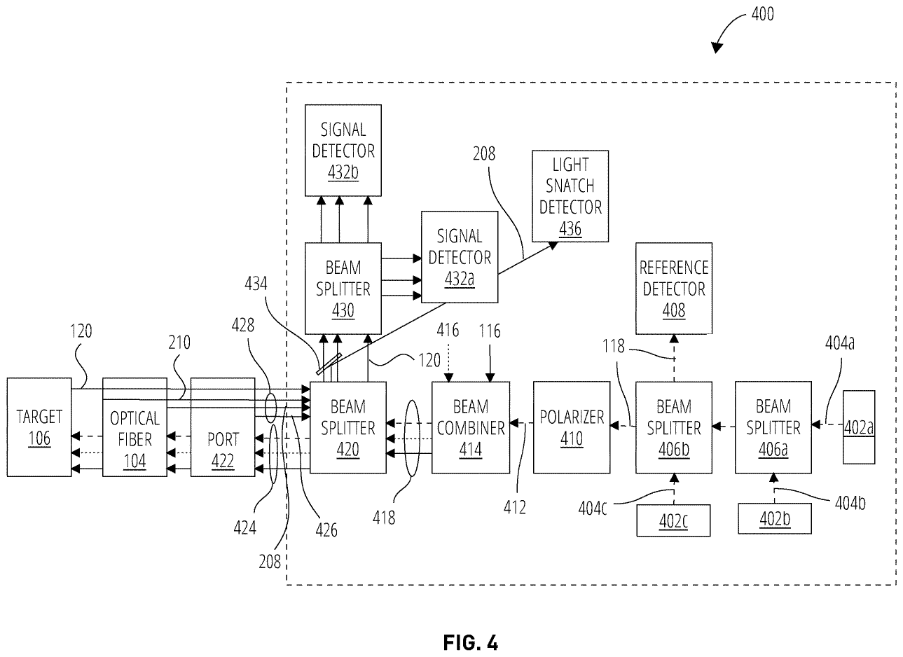

Interrogation sub-system 200 can include an interrogation laser source 202 and optics 204. The interrogation laser source 202 can be configured to generate one or more pulsed laser beams, which can form the interrogation laser pulses 118. With some embodiments (e.g., see FIG. 4), interrogation laser source 202 can be configured to generate interrogation laser pulses 118 having multiple wavelength components.

Optics 204 can be configured to receive the interrogation laser pulses 118 from the interrogation laser source 202 and receive therapeutic laser pulses 116 from a therapeutic laser source (e.g., the therapeutic laser source 108, or the like). The optics 204 can further be configured to combine the therapeutic laser pulses 116 and interrogation laser pulses 118 to form combined laser beam 206 and couple the combined laser beam 206 to the optical fiber 104, such that the combined laser beam 206 can be delivered to the target 106 as described above. The optics 204 can include any of a variety of optical component (e.g., polarizers, beam combiners, collimators, circulators, lenses, etc.) arranged to condition and direct the therapeutic laser pulses 116 and the interrogation laser pulses 118 to the optical fiber 104.

As described above, during operation, some portion of the combined laser beam 206 will be reflected by the proximal end 124 as proximal facet reflection 208 and some portion of the combined laser beam 206 will be reflected by the distal end 126 as distal facet reflection 210. As outlined above, during operation, the proximal end 124 and distal end 126 of the optical fiber 104 will degrade and/or be damaged due to the energy of the combined laser beam 206. More particularly, energy from the therapeutic laser pulses 116, which is part of the combined laser beam 206, will cause damage to the optical fiber 104. The degradation and/or damage of the optical fiber 104 can be interpolated based on the change in the proximal facet reflection 208 and distal facet reflection 210 over time. To that end, the interrogation sub-system 200 further includes a beam splitter 212 and signal detector 214, which can be configured to measure the proximal facet reflection 208 and distal facet reflection 210.

With some embodiments, the beam splitter 212 is disposed between the interrogation laser source 202 and the optics 204. In this arrangement, the beam splitter 212 can be configured to direct the interrogation laser pulses 118 to the optics 204 and direct the proximal facet reflection 208 and distal facet reflection 210 to the signal detector 214. The signal detector 214 can be any of a variety of light detectors. In general, such light detectors may include devices that detect and/or measure characteristics of light beams and encode the detected and/or measured characteristics in electrical signals. Accordingly, the signal detector 214 is configured to measure an intensity of the proximal facet reflection 208 and distal facet reflection 210.

Processing unit 112 can be coupled to signal detector 214 and can execute instructions 128 to receive signals comprising indications of the intensity of proximal facet reflection 208 and distal facet reflection 210. Further, processing unit 112 can execute instructions 128 to interpolate damage to the optical fiber 104. It is to be appreciated, that interrogation sub-system 200 is configured to measure an intensity of the reflected light due to both the proximal facet reflection 208 and the distal facet reflection 210. It is noted that there is not reflection from the target 106 depicted in this figure. It is to be appreciated that during a treatment procedure, there will be many time periods, even short ones, where the target 106 is far enough away (e.g., greater than 4 millimeters (mm), or the like) from the distal end 126 of the optical fiber 104 that any reflection from the target 106 will be insignificant and/or immeasurable. Further, there will be reflections from the optical fiber 104, which can be referred to as internal reflections 216. However, these reflections will remain constant throughout a procedure as the optical fiber 104 should not be damaged and/or degrade during the procedure.

As such, with some embodiments, processing unit 112 can execute instructions 128 to measure the distance 130 and to measure the intensity of proximal facet reflection 208 and distal facet reflection 210 during periods where the distance 130 is greater than a threshold distance (e.g., 4 mm, or the like). During these periods, the output from the signal detector 214 represents only proximal facet reflection 208 and distal facet reflection 210. Further, the processing unit 112 can execute instructions 128 control the surgical laser system 100 based on the signals from the signal detector 214. This is described in greater detail below. However, in general, as the optical fiber 104 degrades and/or is damaged during a procedure, the intensity of the proximal facet reflection 208 and distal facet reflection 210 will decrease. Processing unit 112 can execute instructions 128 to interpolate the amount of damage or degradation the optical fiber 104 has experienced and can control and/or suggest actions based on the interpolated degradation and/or damage.

With some embodiments, the intensity of the proximal facet reflection 208 and distal facet reflection 210 can be measured independently from each other.

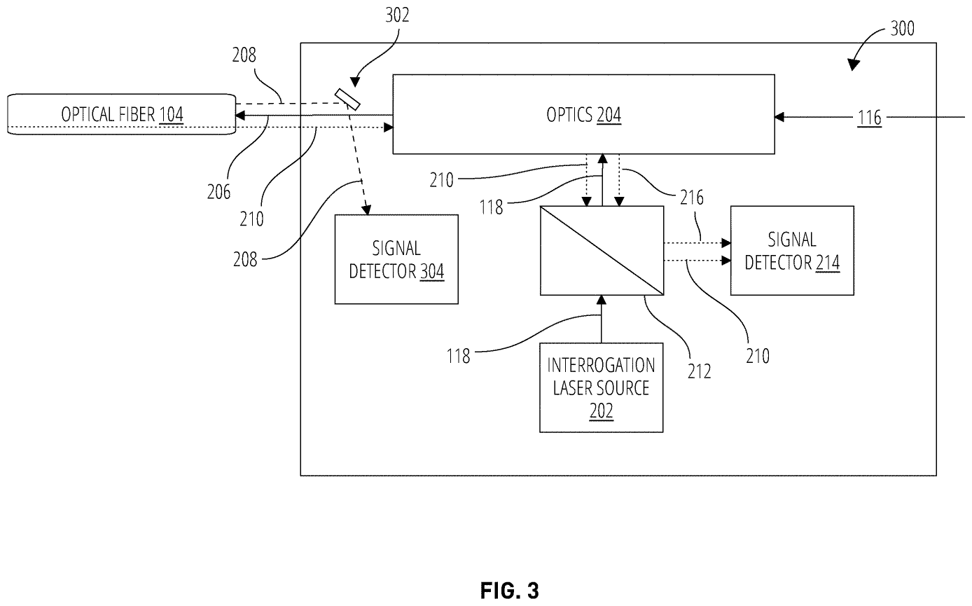

FIG. 3 illustrates an example interrogation sub-system 300, which can be provided in accordance with some embodiments of the present disclosure and used to interpolate optical fiber damage. For example, the interrogation sub-system 300 can be provided as the interrogation sub-system 110 of the surgical laser console 102 shown as part of the surgical laser system 100 in FIG. 1A and FIG. 1B. The interrogation sub-system 300 is like the interrogation sub-system 200 with the difference being that the interrogation sub-system 300 is configured to measure the intensity of the proximal facet reflection 208 and distal facet reflection 210 independently from each other.

The interrogation sub-system 300 includes a light snatch mirror 302 and a signal detector 304. The light snatch mirror 302 is arranged and configured to capture the light reflected only from the proximal end 124 of the optical fiber 104, or proximal facet reflection 208, and direct the proximal facet reflection 208 to the signal detector 304. As the light snatch mirror 302 directs only light reflected from the proximal end 124 of the optical fiber 104, light incident on the signal detector 304 represents only light reflected from the proximal end 124 of the optical fiber 104 and not light reflected from the distal end 126 of the optical fiber 104, the target 106, or even internal reflections from the optics 204. Accordingly, when the optical fiber 104 is not connected to the surgical laser system 100, the intensity level detected by signal detector 304 will be substantially zero.

Further, when a new optical fiber 104 is coupled to the surgical laser system 100, before any therapeutic laser pulses 116 is generated and transmitted through the optical fiber 104, interrogation laser pulses 118 can be generated. In this state, proximal facet reflection 208 captured and directed by the light snatch mirror 302 to the signal detector 304 will be representative of an undamaged optical fiber 104 and can be used as a baseline signal and/or baseline threshold of the proximal facet reflection 208. As the procedure starts and therapeutic laser pulses 116 is delivered through the optical fiber 104, the proximal end 124 of the optical fiber 104 may degrade over time due to incidence of the therapeutic laser pulses 116. As such, the proximal facet reflection 208 may degrade and the intensity of the proximal facet reflection 208 being directed to the signal detector 304 will correspondingly degrade.

Further, as the proximal facet reflection 208 is directed to the signal detector 304 by the light snatch mirror 302, reflections reaching the signal detector 214 will be representative of (1) the distal facet reflection 210, (2) internal reflections 216, and (3) reflections from the target. However, as outlined above, there will be periods during the procedure where the target 106 is far enough (e.g., greater than 4 mm, or the like) away from the distal end 126 of the optical fiber 104, that reflections from the target 106 will be substantially zero.

As such, with some embodiments, processing unit 112 can execute instructions 128 to measure the intensity of the proximal facet reflection 208 via the signal detector 304 and to measure the intensity of the distal facet reflection 210 via the signal detector 214 during periods where the distance 130 is greater than the threshold distance discussed above. Further, the processing unit 112 can execute instructions 128 control the surgical laser system 100 based on the signals from the signal detector 214 and the signal detector 304.

FIG. 4 shows an example interrogation sub-system 400, which can be provided in accordance with some embodiments of the present disclosure. For example, the interrogation sub-system 400 can be provided as the interrogation sub-system 110 of the surgical laser console 102 shown as part of the surgical laser system 100 in FIG. 1A and FIG. 1B. The interrogation sub-system 400 differs from the 200 and 300 in that a specific example of the optical fiber 104 is depicted and further, the interrogation laser source 202 comprises multiple laser sources having different wavelengths.

As depicted, the interrogation sub-system 400 comprises laser sources 402a, 402b, and 402c, which are configured to generate, respectively, laser beams 404a, 404b, and 404c. The laser sources 402a, 402b, and 402c are each configured to generate laser beams of a different wavelengths. For example, laser source 402a can be configured to generate laser beam 404a having a first wavelength while the laser source 402b can be configured to generate the laser beam 404b having a second wavelength different than the first wavelength. Of note, the first wavelength can have an absorption coefficient (e.g., in water, or the like) higher than an absorption coefficient of the second wavelength. Further, the laser source 402c can be configured to generate the laser beam 404c having yet another wavelength, different from the wavelength of the laser beams 404a and 404b.

As used herein, the terms “high” and “low” are intended to be interpreted relative to each other, or in the alternative relative to a threshold characteristic describing the water absorption of a particular wavelength. For example, a high water absorption coefficient can be greater than or equal to 150% of the low water absorption coefficient. In a non-limiting example, the laser beam 404a can have a wavelength of approximately 1310 nanometers (nm); laser beam 404b can have a wavelength of approximately 1340 nm. Further, the laser sources may include a calibration laser beam (e.g., approximately 1431 nm, or the like). This is described in greater detail below.

The laser beams 404a, 404b, and 404c are combined via beam splitters 406a and 406b into interrogation laser beams 408 (e.g., interrogation laser pulses 118 of FIG. 1A, or the like). A reference detector 408 is associated with and in optical communication with the beam splitter 406b. The reference detector 408 is arranged to measure the optical power of each respective component of interrogation laser beams 408 (e.g., component of laser sources 402a, 402b, and 402c).

The beam splitter 406b is further associated with and in optical communication with the polarizer 410. The beam splitter 406b is configured to provide a portion of the interrogation laser beams 408 to the polarizer 410. In some embodiments, the polarity of the polarizer 410 may be pre-configured and arranged to output polarized light beam 412. The interrogation sub-system 400 further includes a beam combiner 414, which is in optical communication with the polarizer 410. In such a manner, the polarized light beam 412 obtained as an output from the polarizer 410 is provided as input to the beam combiner 414. The beam combiner 414 may combine the polarized light beams 412 (e.g., corresponding to the interrogation laser beams 408) with a therapeutic laser beam 416 (e.g., therapeutic laser pulses 116 of FIG. 1A, or the like) and an aiming beam 416, to form combined beam 418.

The interrogation sub-system 400 further includes a beam splitter 420 and a port 422. The beam splitter 420 is arranged in optical communication with the beam combiner 414. As such, the beam splitter 420 can receive the combined beam 418 (comprising the polarized light beam 412, the therapeutic laser beam 416, and the aiming beam 416) from the beam combiner 414 and output the combined beam 418 to the port 422. In such a manner, the beam splitter 420 is optically coupled to the optical fiber 104 via the port 422 such that a portion of the combined beam 418 (e.g., denoted as combined beam portion 424) is transmitted through the optical fiber 104 to be incident on the target 106.

When the combined beam portion 424 is incident on the target 106, the target 106 may reflect some portion of the incident combined beam portion 424 away from the optical fiber 104 and some portion of the light towards the optical fiber 104, wherein the portion of light reflected towards the optical fiber 104 may re-enter the optical fiber 104 (e.g., at the distal end 126 of the optical fiber 104). The portion of the reflected light re-entering the optical fiber 104 at the distal end 126 may be referred to as target reflection beam 426. As noted, the target reflection beam 426 may be contaminated and/or mixed with other reflections. For example, some of the combined beam portion 424 may be reflected from the distal facet (e.g., distal end 126) of the optical fiber 104 as distal facet reflection beam 428 while some of the combined beam portions 424 may be reflected from the proximal facet (e.g., proximal end 124) of the optical fiber 104 as proximal facet reflection beam 430. Further, some of the combined beam portion 424 may be reflected from other internal optical components (e.g., port 422, coupling lenses, etc.) as internal reflection beam 426. The collection of distal facet reflection beam 428, proximal facet reflection beam 430, and internal reflection beam 426 are referred to as parasitic reflected beams 428 (e.g., parasitic reflections 122 of FIG. 1A, or the like).

The present disclosure provides to account for the parasitic reflected beams 428 by either isolating them or measuring them such that the proximal facet reflection beam 430 and the distal facet reflection beam 428 can be measured during a procedure to interpolate damage to the 104 as described herein.

The parasitic reflected beams 428 may be subjected to the beam splitter 420, which directs the parasitic reflected beams 428 to another beam splitter 430. The beam splitter 430 may be in optical communication with signal detectors 432a and 432b. In some examples, the beam splitter 430 may be a polarizing beam splitter. As such portions of parasitic reflected beams 428 having one polarity (e.g., P) may be transmitted to one of the signal detectors 432a or 432b while portions of the parasitic reflected beams 428 having the other polarity (e.g., S) may be transmitted to the other signal detector 432a or 432b.

The interrogation sub-system 400 further includes a light snatch mirror 434 disposed between beam splitter 420 and the beam splitter 430 and configured to direct the proximal facet reflection beam 430 to a light snatch detector 436, thereby eliminating the proximal facet reflection beam 430 from reaching the signal detectors 432a and 432a and as a result interfering with the measurement. The complete details of eliminating the proximal facet reflection beam 430 from the parasitic reflected beams 428 is beyond the scope of this disclosure. However, the general principle relies on the fact that the proximal facet reflection beam 430 have a different angular rotation than other parasitic beams, and as such, can be selectively directed towards light snatch detector 436 by light snatch mirror 434 without interfering with the other components of parasitic reflected beams 428.

Thus, only the remaining components of parasitic reflected beams 428 (e.g., distal facet reflection beam 428 and internal reflection beam 426) as well as the target reflection beam 426 reach the signal detectors 432a and 432b. The signal detectors 432a and 432b are configured to measure intensities of the components of parasitic reflected beams 428 and the target reflection beam 426 and to transmit the intensities to the processing unit 112. Accordingly, the processing unit 112 may estimate the distance 130 between the distal end 126 of the optical fiber 104 and the target 106 based on the measured intensities.

Accordingly, interrogation sub-system 400 provides four (4) optical detectors (e.g., reference detector 408, signal detector 432a, signal detector 432b, and light snatch detector 436). Each of these four (4) detectors can be configured to output a signal indicative of an intensity of a component of the overall laser beams. For example, interrogation sub-system 400 is configured to generate interrogation laser beams 408 comprising laser beams 404a, 404b, and 404c. As such, the four (4) detectors are configured to output a signal for each of the respective laser beams 404a, 404b, and 404c. Further, each of the four (4) detectors are configured to output signals indicative of an intensity of a “dark current” (e.g., leakage current measured when all laser sources 402a, 402b, and 402c) are off. With some embodiments, the four (4) detectors can be 24-bit optical detectors to allow sufficient dynamic range to support a wide range of targets 106 having different reflection characteristics and to support fiber distal end degradation during the treatment procedure. As such, there are sixteen (16) signals contemplated herein (e.g., four (4) from each of the four (4) detectors).

It is to be appreciated that for accurate measurements (1) the interrogation laser beams 408 and the associated parasitic reflected beams 428 should travel through the liquid environment (e.g., water, or the like) to take advantage of the difference in absorption coefficients between the components of the interrogation laser beams 408; and (2) the parasitic reflected beams 428 associated with the interrogation laser beams 408 as well as the contribution from the dark current need to either be eliminated or considered.

It is to be appreciated that internal reflection beam 426 are fixed and do not change with time. As such, these signals can be measured once and the measurements used in an overall distance measurement algorithm. Further it is to be appreciated that the distal facet reflection beam 428 changes during the procedure. For example, the surface of the distal end 126 of the optical fiber 104 is continuously damaged due to the energy impact from the therapeutic laser beam 416 and in instances where fragments of the target 106 impact the surface. Changes to the surface of the distal end 126 of the optical fiber 104 have different, or varying, impacts to each of the different components of the interrogation laser beams 408. Additionally, other phenomenon can contribute to the dynamic nature of the distal facet reflection beam 428, such as, for example, bubbles near the surface of the distal end 126 of the optical fiber 104 forming and collapsing will affect the distal facet reflection beam 428.

Since the distal facet reflection beam 428 changes during the procedure, it cannot be measured upfront like the internal reflection beam 426. Further, the amplitude of the changes in the distal facet reflection beam 428 are often higher than that of the target reflection beam 426. As such, the present disclosure provides to measure the distal facet reflection beam 428 and calibrate and/or adjust the distance measurement algorithm based on the measurement of the distal facet reflection beam 428.

With some embodiments, the processing unit 112 can be configured to execute instructions 128 to measure the distance 130 between the distal end 126 of the optical fiber 104 and the target 106 based on signals received from the reference detector 408, the signal detectors 432a and 432b, and the light snatch detector 436. Examples of measuring the distance 130 are provided in greater detail below.

The disclosure provides to periodically measure the distance 130 and further provides to measure the distal facet reflection beam 428 during periods where the distance 130 between the distal end 126 of the optical fiber 104 and the target 106 is great enough such that all of the interrogation laser beams 408 is absorbed by the liquid medium, and therefore, target reflection beam 426 is substantially zero.

As noted, in a typical lithotripsy procedure, there are many time periods where the target 106 is far enough away from the distal end 126 of the optical fiber 104 (e.g., greater than or equal to 4 millimeters, or the like) such that signals reaching the signal detectors 432a and 432b are originating only from the distal end 126 of the optical fiber 104 (e.g., are only distal facet reflection beam 428). Further, as the internal reflection beam 426 is known and can be subtracted from the parasitic reflected beams 428, the distal facet reflection beam 428 can be derived during these periods.

The processing unit 112 can execute instructions 128 to identify periods where the distance 130 is above a threshold value (e.g., 4 mm, or the like) for a long enough time and can measure the distal facet reflection beam 428 at this time and update and/or calibrate the overall distance 130 measurement algorithm based on this measurement. Further, this distal facet reflection beam 428 can be measured over time and the change in the distal facet reflection beam 428 over time used to interpolate damage to the distal end 126 of the optical fiber 104.

Additionally, as the proximal facet reflection beam 430 is isolated by the light snatch mirror 434, the intensity of the proximal facet reflection beam 430 can be measured and the change over time can be used to interpolate the damage to the proximal end 124 of the 104.

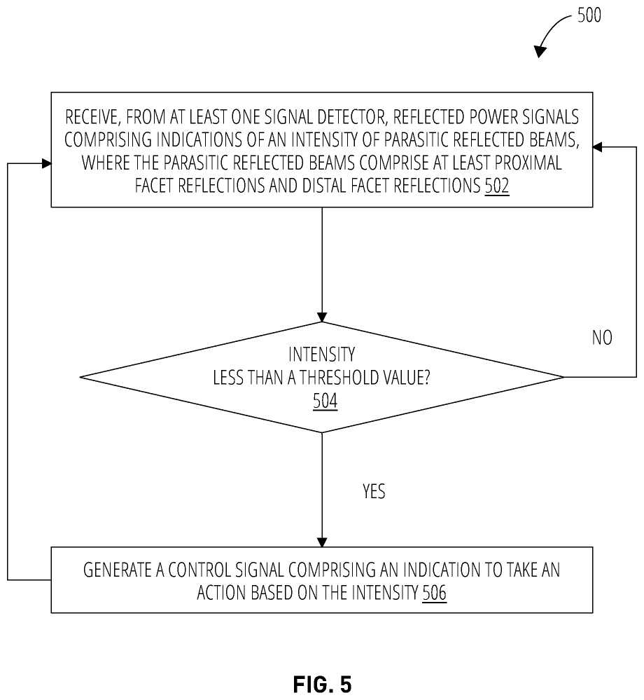

FIG. 5 illustrates a flowchart showing a method 500 for interpolating damage to an optical fiber and for controlling a surgical laser system based on the interpolated damage. The method 500 can be implemented during a surgical laser procedure, such as a laser lithotripsy procedure. The method 500 is described with reference to the surgical laser system 100 of FIG. 1A and FIG. 1B, the interrogation sub-system 200 of FIG. 2, the interrogation sub-system 300 of FIG. 3, and to the interrogation sub-system 400 of FIG. 4. It is to be appreciated however, that the method 500 could be implemented by a surgical system different than the one depicted herein.

The method 500 can begin at block 502. At block 502 “receive, from at least one signal detector, reflected power signals comprising indications of an intensity of parasitic reflected beams, where the parasitic reflected beams comprise at least proximal facet reflections and distal facet reflections” reflected power signals comprising indications of an intensity of parasitic reflected beams can be received, where the parasitic reflected beams comprise at least proximal facet reflections and distal facet reflections. For example, processing unit 112 can execute instructions 128 to receive from signal detector 214, signals comprising indications of the intensity (or power) of at least proximal facet reflection 208 and distal facet reflection 210. In other embodiments, processing unit 112 can execute instructions to receive, signals comprising indications of the intensity of proximal facet reflection 208 from signal detector 304 and signals comprising indications of the intensity of distal facet reflection 210 from the signal detector 214. In yet other embodiments, processing unit 112 can execute instructions 128 to receive signals comprising indications of the intensity of proximal facet reflection 208 from the light snatch detector 436 and signals comprising indications of the intensity of distal facet reflection 210 from the signal detector 432a and/or signal detector 432b. As noted above, in some embodiments, signals comprising indications of the distal facet reflection 210 will include indications of other parasitic reflections (e.g., internal reflection beam 426, or the like). Further, with some embodiments, multiple signal measurements (e.g., a time series of signals, or the like) from the detectors can be received.

Further, as introduced above there will be periods of time during a procedure where the target 106 is far enough away from the distal end 126 of the optical fiber 104 that all of interrogation laser pulses 118 will be absorbed by the liquid medium, and as such, target reflected laser pulses 120 will be substantially zero, or will be small enough such that the reflected light incident on the signal detectors 432a and 432b will only be representative of the distal facet reflection 210 and internal reflection beam 426. With some embodiments, processing unit 112 can execute instructions 128 to receive signals from the signal detectors (e.g., signal detectors 214, 304, 432a, 432b, and/or 436) during periods where the target 106 is far enough away such that the received signals are only dependent on parasitic reflections as outlined above. Further, as outlined above, in some embodiments the reflections can be based on an incident laser beam having multiple wavelength components (e.g., laser beams 404a, 404b, 404c, etc.). As such, signals received from the signal detectors can correspond to these multiple wavelength components.

Additionally, with some embodiments, processing unit 112 can execute instructions 128 to receive signals from signal detectors comprising indications of “dark current.” For example, where the therapeutic laser pulses 116 and interrogation laser pulses 118 are off, the detectors (e.g., signal detectors 214, 304, 432a, 432b, and/or 436) will measure some light intensity, which is referred to herein as “dark current” intensity. Processing unit 112 can execute instructions 128 to measure this dark current and interpolate damage to the optical fiber 104 based on the intensity of the dark current.

Continuing to decision block 504 “intensity less than a threshold value?” a determination can be made as to whether the intensity (e.g., intensity of the reflected signals) is less than a threshold value. For example, processing unit 112 can execute instructions 128 to determine whether the intensity of the signals received from the signal detectors are less than a threshold value. With some embodiments, the threshold value can be preset. With other examples, processing unit 112 can execute instructions 128 to determine the threshold at the start of each procedure when the optical fiber 104 is new and before it has been exposed to therapeutic laser pulses 116. For example, processing unit 112 can execute instructions 128 to measure the parasitic reflections (e.g., proximal facet reflection 208, distal facet reflection 210, internal reflection beam 426, etc.) and determine a baseline level of the reflections. Further, processing unit 112 can execute instructions 128 to set the threshold based on this baseline value, such as, for example by setting the threshold as the baseline multiple by a constant value. For example, for the proximal facet reflection 208 and/or distal facet reflection 210, processing unit 112 can execute instructions 128 to set the threshold as the baseline value multiplied by a constant less than 1 (e.g., 0.9, 0.8, 0.7, 0.6, 0.5, etc.) As another example, for the dark current value, processing unit 112 can execute instructions 128 to set the threshold as the baseline value multiplied by a constant greater than 1 (e.g., 1.25, 1.5, 1.75, 2.0, 3.0, 4.0, etc.)

From decision block 504, method 500 can continue to block 506 or return to block 502. For example, where a determination is made that the intensity is less than the threshold value, the method 500 can continue from decision block 504 to block 506 while the method 500 can return to block 502 from decision block 504 where a determination is made that the intensity is not less than the threshold value.

At block 506 “generate a control signal comprising an indication to take an action based on the intensity” a control signal comprising an indication to take an action based on the intensity can be generated. For example, processing unit 112 can execute instructions 128 to generate a control signal comprising an indication for the surgical laser system 100 to take some action based on the intensity. In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 208 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 422, damage to a blast shield, or the like). As another example, a small (e.g., 5%, 10%, 15%, 20%, 25%, or the like) decrease in the intensity of the proximal facet reflection 208 and/or distal facet reflection 210 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104.

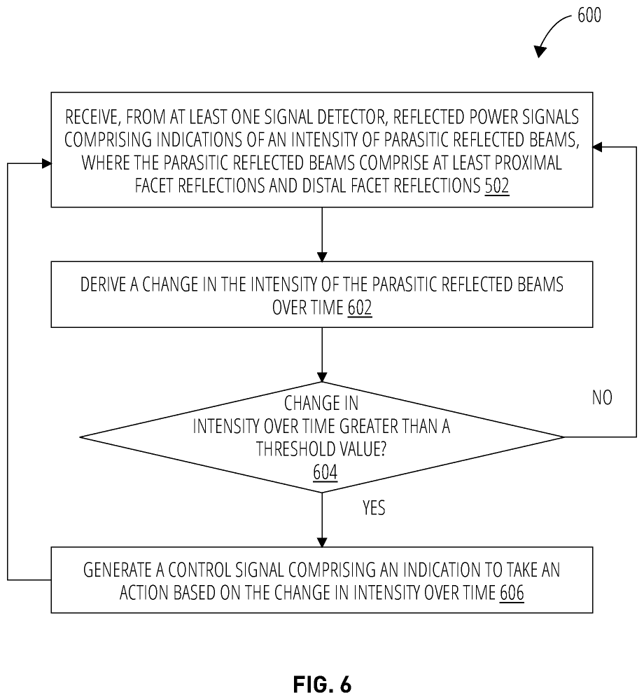

FIG. 6 illustrates a flowchart showing a method 600 for interpolating damage to an optical fiber and for controlling a surgical laser system based on the interpolated damage. The method 600 can be implemented during a surgical laser procedure, such as a laser lithotripsy procedure. The method 600 is described with reference to the surgical laser system 100 of FIG. 1A and FIG. 1B, the interrogation sub-system 200 of FIG. 2, the interrogation sub-system 300 of FIG. 3, and to the interrogation sub-system 400 of FIG. 4. It is to be appreciated however, that the method 600 could be implemented by a surgical system different than the one depicted herein. Further, the method 600 references operations or blocks from the method 500 of FIG. 5. This is done for convenience.

The method 600 can begin at block 502 and continue to block 602. At block 602 “derive a change in the intensity of the parasitic reflected beams over time” an intensity over time of the parasitic reflected beams can be determined. For example, processing unit 112 can execute instructions 128 to determine an intensity of the proximal facet reflection 208, distal facet reflection 210 and/or dark current reflections over a time period (e.g., hundreds of seconds, minutes, or the like). As a specific example, processing unit 112 can execute instructions 128 to determine a change in of the intensity of the proximal facet reflection 208 and/or distal facet reflection 210 over a time period of 240 seconds, 360 seconds, between 120 and 600 seconds. With some embodiments, processing unit 112 can execute instructions 128 to determine a change in the intensity of the proximal facet reflection 208 and/or distal facet reflection 210 over time and to further derive a slope, or rate of the change, over the time period.

Continuing to decision block 604 “change in intensity over time greater than a threshold value?” a determination can be made as to whether the change in intensity over time is greater than a threshold value. For example, processing unit 112 can execute instructions 128 to determine whether the change in the intensity of the proximal facet reflection 208 and/or distal facet reflection 210 over time is greater than a threshold value. Further, where at block 602 the slope or rate of chance is derived, processing unit 112 can execute instructions 128 to determine whether the slope is greater than a threshold value. With some embodiments, the threshold values can be preset.

From decision block 604, method 600 can continue to block 606 or return to block 502. For example, where a determination is made that the change in intensity over time (or the slope of the change) is greater than the threshold value the method 600 can continue from decision block 604 to block 606 while the method 600 can return to block 502 from decision block 604 where a determination is made that the change in intensity over time (or the slope of the change) is not greater than the threshold value.

At block 606 “generate a control signal comprising an indication to take an action based on the change in intensity over time” a control signal comprising an indication to take an action based on the change in intensity over time can be generated. For example, processing unit 112 can execute instructions 128 to generate a control signal comprising an indication for the surgical laser system 100 to take some action based on the change in intensity (or the slope of the change). In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 208 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 422, damage to a blast shield, or the like). As another example, a small (e.g., 5%, 10%, 15%, 20%, 25%, or the like) decrease in the intensity of the proximal facet reflection 208 and/or distal facet reflection 210 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104. As another example, where the dark current reading increases above the baseline for a long enough time, it may indicate that the distal end 126 of the optical fiber 104 is burnt and should be cleaved to maintain transmission efficiency of the optical fiber 104.

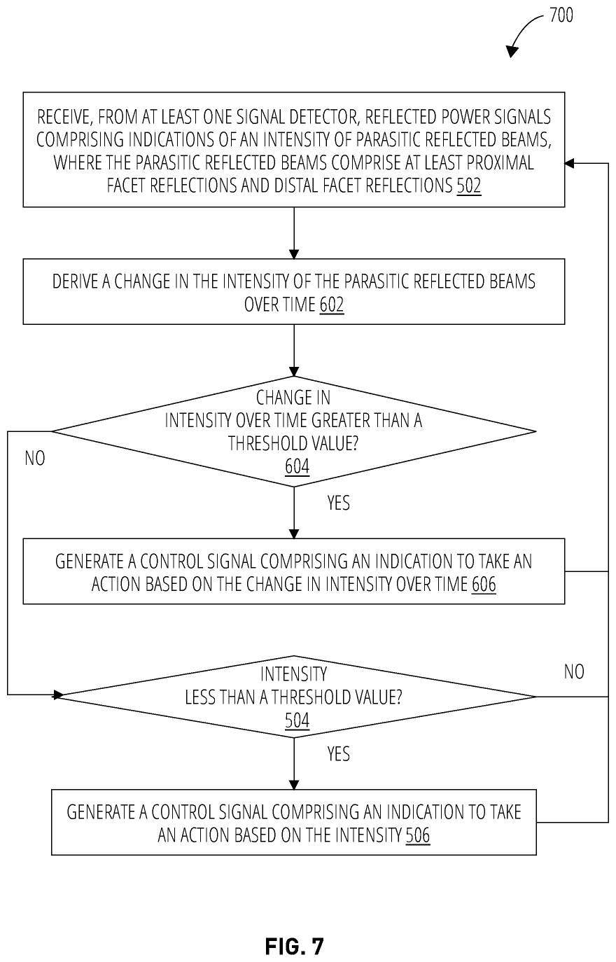

FIG. 7 illustrates a flowchart showing a method 700 for interpolating damage to an optical fiber and for controlling a surgical laser system based on the interpolated damage. The method 700 can be implemented during a surgical laser procedure, such as a laser lithotripsy procedure. The method 600 is described with reference to the surgical laser system 100 of FIG. 1A and FIG. 1B, the interrogation sub-system 200 of FIG. 2, the interrogation sub-system 300 of FIG. 3, and to the interrogation sub-system 400 of FIG. 4. It is to be appreciated however, that the method 700 could be implemented by a surgical system different than the one depicted herein. Further, the method 700 references operations or blocks from the method 500 of FIG. 5 and the method 600 of FIG. 6. This is done for convenience.

The method 700 can begin at block 502 and continue to block 602 and then decision block 604. For decision block 604, method 700 can continue to either block 606 or decision block 504. For example, method 700 can continue from decision block 604 to block 606 where a determination is made that the change in intensity over time (or the slope of the change) is greater than the threshold value while method 700 can continue from decision block 604 to decision block 504 where a determination is made that the change in intensity over time (or the slope of the change) is not greater than the threshold value. From block 606, method 700 can return to block 502. Additionally, the method 700 can continue from decision block 504 to either block 506 or return to block 502. For example, method 700 can continue from decision block 504 to block 506 where a determination is made that the intensity is less than the threshold value while method 700 can continue from decision block 504 to block 502 where a determination is made that the intensity is not less than the threshold value.

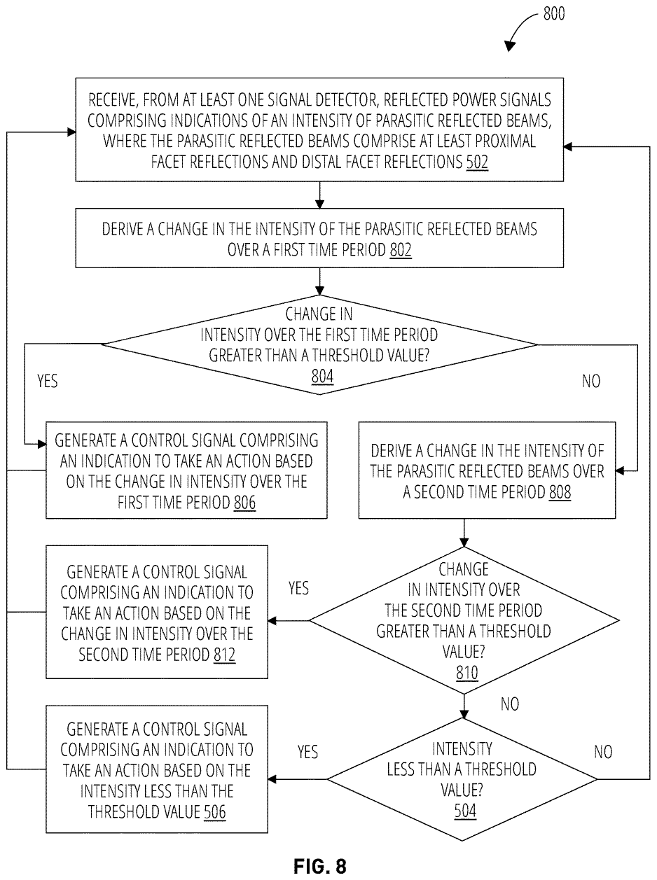

FIG. 8 illustrates a flowchart showing a method 800 for interpolating damage to an optical fiber and for controlling a surgical laser system based on the interpolated damage. The method 800 can be implemented during a surgical laser procedure, such as a laser lithotripsy procedure. The method 800 is described with reference to the surgical laser system 100 of FIG. 1A and FIG. 1B, the interrogation sub-system 200 of FIG. 2, the interrogation sub-system 300 of FIG. 3, and to the interrogation sub-system 400 of FIG. 4. It is to be appreciated however, that the method 800 could be implemented by a surgical system different than the one depicted herein. Further, the method 600 references operations or blocks from the method 500 of FIG. 5. This is done for convenience.

The method 800 can begin at block 502 and continue to block 802. At block 802 “derive a change in the intensity of the parasitic reflected beams over a first time period” an intensity over a first time period of the parasitic reflected beams can be determined. For example, processing unit 112 can execute instructions 128 to determine an intensity of the proximal facet reflection 208, distal facet reflection 210 and/or dark current reflections over a first time period (e.g., hundreds of seconds, minutes, or the like). As a specific example, processing unit 112 can execute instructions 128 to determine a change in of the intensity of the proximal facet reflection 208 and/or distal facet reflection 210 over a time period of 240 seconds, 360 seconds, between 120 and 600 seconds. With some embodiments, processing unit 112 can execute instructions 128 to determine a change in the intensity of the proximal facet reflection 208 and/or distal facet reflection 210 over time and to further derive a slope, or rate of the change, over the time period.

Continuing to decision block 804 “change in intensity over the first time period greater than a threshold value?” a determination can be made as to whether the change in intensity over the first time period is greater than a threshold value. For example, processing unit 112 can execute instructions 128 to determine whether the change in the intensity of the proximal facet reflection 208 and/or distal facet reflection 210 over the first time period is greater than a threshold value. Further, where at block 602 the slope or rate of chance is derived, processing unit 112 can execute instructions 128 to determine whether the slope is greater than a threshold value. With some embodiments, the threshold values can be preset.

From decision block 804, method 800 can continue to block 806 or block 808. For example, where a determination is made that the intensity over the first time period (of the slope) is greater than the threshold value the method 800 can continue from decision block 804 to block 806 while the method 800 can continue from decision block 804 to block 808 where a determination is made that the intensity over the first time period (of the slope) is greater than the threshold value.

At block 806 “generate a control signal comprising an indication to take an action based on the change in intensity over the first time period” a control signal comprising an indication to take an action based on the change in intensity over the first time period can be generated. For example, processing unit 112 can execute instructions 128 to generate a control signal comprising an indication for the surgical laser system 100 to take some action based on the change in intensity (or the slope of the change) over the first time period. In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 208 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 422, damage to a blast shield, or the like). As another example, a small (e.g., 5%, 10%, 15%, 20%, 25%, or the like) decrease in the intensity of the proximal facet reflection 208 and/or distal facet reflection 210 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104. As another example, where the dark current reading increases above the baseline for a long enough time, it may indicate that the distal end 126 of the optical fiber 104 is burnt and should be cleaved to maintain transmission efficiency of the optical fiber 104.

At block 808 “derive a change in the intensity of the parasitic reflected beams over a second time period” an intensity of the parasitic reflected beams over a second time period, different from the first time period, can be determined. For example, processing unit 112 can execute instructions 128 to determine an intensity of the proximal facet reflection 208, distal facet reflection 210 and/or dark current reflections over a second time period (e.g., hundreds of seconds, minutes, or the like) where the second time period is different from the first time period. As a specific example, processing unit 112 can execute instructions 128 to determine a change in of the intensity of the proximal facet reflection 208 and/or distal facet reflection 210 over a time period of 240 seconds, 360 seconds, between 120 and 600 seconds. With some embodiments, processing unit 112 can execute instructions 128 to determine a change in the intensity of the proximal facet reflection 208 and/or distal facet reflection 210 over time and to further derive a slope, or rate of the change, over the time period.

Continuing to decision block 810 “change in intensity over the second time period greater than a threshold value?” a determination can be made as to whether the change in intensity over the second time period is greater than a threshold value. For example, processing unit 112 can execute instructions 128 to determine whether the change in the intensity of the proximal facet reflection 208 and/or distal facet reflection 210 over the second time period is greater than a threshold value. Further, where at block 602 the slope or rate of chance is derived, processing unit 112 can execute instructions 128 to determine whether the slope is greater than a threshold value. With some embodiments, the threshold values can be preset.

From decision block 810, method 800 can continue to block 812 or decision block 504. For example, where a determination is made that the intensity over the second time period (of the slope) is greater than the threshold value the method 800 can continue from decision block 810 to block 812 while the method 800 can continue from decision block 810 to decision block 504 where a determination is made that the intensity over the second time period (of the slope) is greater than the threshold value.

At block 812 “generate a control signal comprising an indication to take an action based on the change in intensity over the second time period” a control signal comprising an indication to take an action based on the change in intensity over the second time period can be generated. For example, processing unit 112 can execute instructions 128 to generate a control signal comprising an indication for the surgical laser system 100 to take some action based on the change in intensity (or the slope of the change) over the second time period. In some embodiments, processing unit 112 can execute instructions 128 to generate a control signal to cause a change in operation of the surgical laser system 100 (e.g., increase pulse frequency of therapeutic laser pulses 116, increase pulse energy of therapeutic laser pulses 116, increase pulse frequency and energy of therapeutic laser pulses 116, change a suggested working distance based on measuring distance 130, or stop generation of the therapeutic laser pulses 116) to compensate for the degradation of the optical fiber 104 or to prevent damage to the surgical laser system 100. With other embodiments, processing unit 112 can execute instructions 128 to generate a control signal to cause the surgical laser system 100 to display an alert to the user (e.g., an alert suggesting a change in operating parameters such as pulse frequency and/or energy, an alert suggesting the user cleave the distal end 126 of the optical fiber 104 to renew the optical fiber 104, or an alert indicating a reduction is lasing efficiency due to degraded optical fiber 104).

For example, a large (e.g., 40%, 50%, 60%, or the like) decrease in the intensity of the proximal facet reflection 208 from the baseline can indicate severe damage to the proximal end 124 of the optical fiber 104, which can be indicative of a “dead” optical fiber that does not transmit enough laser energy. Continued lasing with such an optical fiber is not efficient and may result in damage to the surgical laser system 100 (e.g., damage to the port 422, damage to a blast shield, or the like). As another example, a small (e.g., 5%, 10%, 15%, 20%, 25%, or the like) decrease in the intensity of the proximal facet reflection 208 and/or distal facet reflection 210 from the baseline can indicate damage to the proximal end 124 and/or distal end 126 of the optical fiber 104, which may result in a corresponding decrease in the energy of the therapeutic laser pulses 116 delivered from the optical fiber 104. As such, adjusting the operational parameters of the surgical laser system 100 (e.g., corresponding increase in pulse frequency and/or energy of the therapeutic laser pulses 116) may result in a consistent delivery of energy of therapeutic laser pulses 116 ultimately delivered from the optical fiber 104. As another example, where the dark current reading increases above the baseline for a long enough time, it may indicate that the distal end 126 of the optical fiber 104 is burnt and should be cleaved to maintain transmission efficiency of the optical fiber 104.

From decision block 504, the method 800 can continue to either block 506 or return to block 502. For example, method 800 can continue from decision block 504 to block 506 where a determination is made that the intensity is less than the threshold value while method 800 can return from decision block 504 to block 502 where a determination is made that the intensity is not less than the threshold value. Further, from block 806 and block 812, the method 800 can return to block 502.

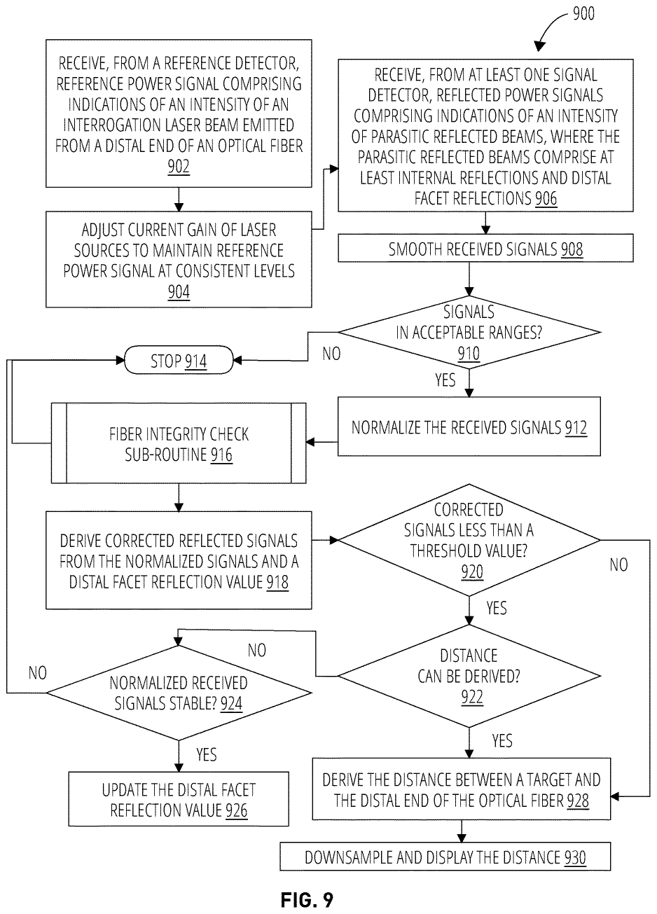

FIG. 9 illustrates a flowchart showing a method 900 for interpolating a damage to an optical fiber as part of measuring a distance between a distal end of an optical fiber a target. The method 900 can be implemented during a surgical laser procedure, such as a laser lithotripsy procedure. The method 900 is described with reference to the surgical laser system 100 of FIG. 1A and FIG. 1B and to the interrogation sub-system 400 of FIG. 4. It is to be appreciated however, that the method 900 could be implemented by a surgical system different than the one depicted herein.

The method 900 can begin at block 902. At block 902 “receive, from a reference detector, reference power signal comprising indications of an intensity of an interrogation laser beam emitted from a distal end of an optical fiber” a reference power signal comprising indications of an intensity of an interrogation laser beam emitted from a distal end of an optical fiber can be received. For example, processing unit 112 can execute instructions 128 to receive from reference detector 408, signals comprising indications of the power of interrogation laser pulses 118. As noted above, interrogation laser pulses 118 can comprise multiple components (e.g., laser beams 404a, 404b, 404c, etc.) In some examples, processing unit 112 can execute instructions 128 to receive indications of a power of each respective component.

Continuing to block 904 “adjust current gain of laser sources to maintain reference power signal at consistent levels” the current gain of laser sources can be adjusted to maintain reference power signals at consistent levels. For example, processing unit 112 can execute instructions 128 to adjust the current gain of the laser sources 402a, 402b, and 402c so that the signals output from the reference detector 408 corresponding to these laser sources are maintained at consistent levels.

Continuing to block 906 “receive, from at least one signal detector, reflected power signals comprising indications of an intensity of parasitic reflected beams, where the parasitic reflected beams comprise at least internal reflections and distal facet reflections” reflected power signals comprising indications of an intensity of parasitic reflected beams can be received, where the parasitic reflected beams comprise at least internal reflections and distal facet reflections. For example, processing unit 112 can execute instructions 128 to receive from signal detectors 432a and/or 432b, signals comprising indications of the power of parasitic reflected beams 428. As noted above, parasitic reflected beams 428 can comprise multiple components (e.g., distal facet reflection 210, proximal facet reflection 208, internal reflection beam 426, etc.) In some examples, proximal facet reflection 208 can be isolated from the parasitic reflected beams 428 (e.g., by light snatch mirror 434 and light snatch detector 436, or the like). As such, signals generated by signal detectors 432a and 432b will depend only on distal facet reflection 210 and internal reflection beam 426. Further, as introduced above there will be periods of time during a procedure where the target 106 is far enough away from the distal end 126 of the optical fiber 104 that all of interrogation laser pulses 118 will be absorbed by the liquid medium, and as such, target reflected laser pulses 120 will be substantially zero, or will be small enough to allow calibration as described herein.

Continuing to block 908 “smooth received signals” the received signals can be smoothed. For example, processing unit 112 can execute instructions 128 to apply a filter to reduce noise. With some embodiments, processing unit 112 can execute instructions 128 to apply a first order low pass filter (LPF) to the received signals. However, it is to be appreciated that other types of smoothing algorithms may be applied to filter and/or reduce noise in the received signals. For example, smoothing the received signals via a LPF may facilitate elimination of high frequency noise that may appear due to operation of the therapeutic laser pulses 116, movement of the distal end 126 of the optical fiber 104, electronic noise, or any other physical phenomena that may affects or contaminate the received signals.

Processing unit 112 can execute instructions 128 to derive a filtered (or smoothed) received signal for each of the sixteen (16) signals received at blocks block 902 and block 906. The filtered received signal (Signal_{filtered}) can be derived based on the following equation, where α is the smoothing constant and is set between zero (0) and one (1) with one (1) equaling no smoothing, Signal is the raw signal, PriorSignal_{filtered} is the filtered signal derived at the last time (or sampling) period.

$${Signal}_{filtered} = ( 1 - α ) {PriorSignal}_{filtered} + α ( Signal )$$

Continuing to decision block 910 “signals in acceptable ranges?” a determination can be made as to whether the signals are in acceptable ranges. For example, processing unit 112 can execute instructions 128 to determine whether the signals received at blocks block 902 and block 906 are within a raw signal threshold range of values. From decision block 910, method 900 can continue to block 912 or done block 914. For example, where a determination is made that the received signals are within the raw signal threshold range of values, the method 900 can continue from decision block 910 to block 912. Alternatively, where a determination is made that any one of the received signals are not within the raw signal threshold range of values, the method 900 can continue from decision block 910 to done block 914. At done block 914, method 900 can terminate. In some embodiments, processing unit 112 can execute instructions 128 to generate an error and display the error on a display screen associated with the interrogation sub-system 400.

At block 912 “normalize the received signals” the received signals (e.g., the signals received at block 902 and block 906) can be normalized. For example, processing unit 112 can execute instructions 128 to normalize the received signals to remove contributions to the signal from dark current. In general, processing unit 112 executes instructions 128 to derive normalized signals to eliminate, from the received signals, bias not related to the measured signals. For example, processing unit 112 can execute instructions 128 to derive normalized signals for each component of the interrogation laser pulses 118 (e.g., laser beams 404a, 404b, and 404c) based on removing contributions to each signal from the dark current and then normalizing the signals of each the three (3) signal detectors with the signal from the reference detector 408.

Accordingly, the signals are normalized by subtracting the filtered dark current signals of each of the four (4) detectors from each of the other 12 filtered signals respectively. These signals can be referred to as the “unbiased” signals. Subsequently, the unbiased signals for each laser source (e.g., 1310 nm source, 1340 nm source, 1431 nm source, etc.) from the three (3) signal detectors are divided by the respective unbiased signal from the reference detector to compensate for any laser source intensity fluctuations and to eliminate any dependency in laser emitting power. As such, there are nine (9) normalized signals (e.g., one for each component of interrogation laser pulses 118 and each of the three (3) signal detector 432a, signal detector 432b, and light snatch detector 436).

Continuing to subroutine block 916 “fiber integrity check sub-routine” a sub-routine to check the integrity of the optical fiber 104 can be executed. For example, processing unit 112 can execute instructions 128 to apply a sub-routine configured to check the integrity of the optical fiber 104. Said differently, processing unit 112 can execute instructions 128 to interpolate the damage to the optical fiber 104 and apply an action based on the interpolation. With some embodiments, processing unit 112 can execute instructions 128 to apply any one of the methods 500, 600, 700, or 800 or FIG. 5, FIG. 6, FIG. 7, or FIG. 8, respectively at subroutine block 916. With some embodiments, the normalized signals for the proximal facet reflection 208 and the distal facet reflection 210 derived at block 912 can be used in the subroutine block 916. Further, the dark current signals discussed above can be used. In such a manner, damage to either the proximal end 124 or the distal end 126 of the optical fiber 104 can be interpolated based on these signals and/or a change of these signals over time as discussed above.

Continuing to block 918 “derive correct reflected signals from the normalized signals and a distal facet reflection value” corrected reflected power signals can be generated based on the normalized reflected power signals and a distal facet reflection value. As used herein, the distal reflection value is the derived value of the contribution of the distal facet reflection 210, which is repeatedly updated as described herein. In general, corrected reflected power signals are derived based on subtracting both the internal reflections and the last known fiber distal end reflection normalized signals from the normalized signals associated with components of the interrogation laser pulses 118 having low water absorption coefficients (e.g., the 1310 nm and 1340 nm laser sources, or the like). As such, the resulted signals represent reflections only from the target (e.g., target reflected laser pulses 120) and eliminates internal reflections (e.g., internal reflection beam 426) and reflections from the distal end 126 of the optical fiber 104 (e.g., distal facet reflection 210). It is noted that the subtraction is done twice, once without considering the normalized signals associated with the calibration component of the interrogation laser pulses 118 (e.g., the 1431 nm laser source, or the like) and once with correcting for the current known distal facet reflection values for the high and low water absorption components of the interrogation laser pulses 118.

Further, the correction is conducted using the ratio between the normalized signal of the calibration component signal (e.g., the 1431 nm laser signal) and its last known fiber distal end reflection normalized signals (e.g., the distal facet reflection value). This is to compensate for changes in the reflections from the distal end 126 (e.g., distal facet reflection 210) that are represented by the calibration component but not incorporated yet into the last known distal facet reflection values for the high and low absorption components.

With some embodiments, processing unit 112 can execute instructions 128 to derive corrected reflected power signals based on subtracting the product of the distal facet reflection values for each of the high and low water absorption components (e.g., 1340 nm and 1310 nm) and a ratio of the normalized signal for the calibration component (e.g., 1410 nm) and the distal facet reflection values for the calibration component (e.g., 1410 nm) from the normalized signals for the high and low water absorption components (e.g., 1340 nm and 1310 nm), which are based on signals from one or more of the detectors 432a or 432b as outlined above.

Further, processing unit 112 can execute instructions 128 to derive corrected reflected power signals that do not account for the calibration component based on a difference between the normalized signals and the distal facet reflection values.

Accordingly, output from block 918 is four (4) normalized corrected signals associated with the high and low water absorption components (e.g., 1340 nm and 1310 nm) of the interrogation laser pulses 118 and arriving at one of the signal detectors (e.g., signal detector 432a or signal detector 432b), two (2) of them considering the calibration component and two (2) without considering the calibration component.

Continuing to decision block 920 “corrected signals less than a threshold value?” a determination is made as to whether the corrected signals derived at block 918 are less than a threshold value. For example, processing unit 112 can execute instructions 128 to determine whether any one of the derived corrected signals are less than a threshold value, such as, corrected signal threshold values. In some embodiments, the corrected signal threshold values can include a value for each of the selected components of the reflected power signals.

Said differently, each one of the corrected signals derived at block 918 is checked to verify if distance should be calculated. For example, if the value of any one of the corrected signals is too low (e.g., less than the threshold), the distance 130 is automatically considered far enough from the target without further calculations. The threshold is either a predefined absolute minimum necessary to derive distance or relative to the last known distal facet reflection value. This is done to avoid distance calculation with very low signals, since these are very sensitive to noise and the calculated results may vary between the two extremes of contact and long distance, although the actual distance is constant. The assumption behind this is that low signals are associated with greater distances (e.g., greater than 4 mm, or the like).

From decision block 920, method 900 can continue to decision block 922 or block 928. For example, where a determination is made that the corrected signals are less than the threshold value, method 900 can continue from decision block 920 to decision block 922; while where a determination is made that the corrected signals are not less than the threshold value, method 900 can continue from decision block 920 to block 928. In some embodiments, only where a determination is made than all of the corrected signals are not less than the threshold value will the method 900 continue from decision block 920 to block 928.

At decision block 922 “distance can be derived?” a determination can be made as to whether the distance can be derived. Said differently, a determination can be made as to whether the expected calculated distance is close enough to the target 106, but not in contact with the target 106. For example, if the expected calculated distance is above a predefined threshold distance, the distance is automatically considered far enough from the target (max distance) without further calculations. This is the verification associated with decision block 920. On the other hand, if the expected calculated distance is below a predefined threshold distance, the distance is automatically considered as contact without further calculations. This is done to avoid negative distance calculations, which are not real.

Processing unit 112 can execute instructions 128 to determine whether the corrected reflected signals are within ranges suitable for deriving the distance 130 based on the following pseudo code:

IF: the distal facet reflection value is less than or equal to the threshold (e.g., product of the
threshold and the current distal facet reflection value)
THEN: the distance is the maximum distance
ELSE IF: a product of a ratio of the distal facet reflection values for the high and low
water absorption coefficients and DetFactor (e.g., an empirical correction factor to account for
differences in the spectral response of the signal detector between the different wavelengths) is
greater than or equal to a first signal ratio threshold AND the corrected distal fact reflection
value is less than or equal to a the distal facet reflection value threshold
THEN: the distance is the maximum distance
ELSE IF: the product of the ratio of the distal facet reflection values for the high and
low water absorption coefficients and DetFactor is less than or equal to a second signal ratio
threshold that is less than the first signal ration threshold
THEN: the distance is the maximum distance
ELSE: the distance equals zero (0)

With some examples, the maximum distance for calculation can be 3 mm, 4 mm, 5 mm, 6 mm, 7 mm, 8 mm, or the like).

From decision block 922, method 900 can continue to decision block 924 or block 928. For example, where a determination is made that the distance can be derived, method 900 can continue from decision block 922 to block 928; while where a determination is made that the distance cannot be derived, the method 900 can continue from decision block 922 to decision block 924.

At decision block 924 “normalized received signals stable?” a determination can be made as to whether the normalized received signals are stable. For example, processing unit 112 can execute instructions 128 to determine whether the derived standard deviation of the normalized reflected power signals is less than a stability threshold value. From decision block 924, method 900 can continue to block 926 or return to block 902 (not shown). For example, where a determination is made that the normalized received signals are stable, the method 900 can continue from decision block 924 to block 926; while where a determination is made that the normalized received signals are not stable, the method 900 can continue from decision block 924 to done block 914.

At block 926 “update the distal facet reflection value” the distal facet reflection value can be updated. For example, processing unit 112 can execute instructions 128 to derive an updated distal facet reflection value to calibrate the interrogation sub-system 400 as outlined herein. As a specific example, processing unit 112 can execute instructions 128 to update the distal facet reflection value based on the prior measured signal minus the internal reflection value for each component of the reflected signal (e.g., 1310 nm, 1340 nm, 1431 nm, or the like).

At block 928 “derive the distance between a target and the distal end of the optical fiber” a distance between the target and the distal end of the optical fiber can be derived. Said differently, where the estimated distance 130 between the distal end 126 of the optical fiber 104 and the target 106 is not an edge case (e.g., zero (0), Max, etc.) then the distance can be derived according to the fundamental physical equation incorporating the difference between water absorption coefficients of two different wavelengths (e.g., 1340 nm (high) and 1310 nm (low)). This is done twice, once for the distance considering the calibration component (e.g., 1431 nm) and once for the distance not considering the calibration component.

For example, processing unit 112 can execute instructions 128 to derive the distance 130 between the distal end 126 of the optical fiber 104 and the target 106 based on the following the ratio of the corrected and normalized signals for the high and low water absorption components.

It will further be appreciated that two distances D will be derived based on the above pseudo code. For example, D considering the calibration component (e.g., 1431 nm) and D without. Processing unit 112 can execute instructions 128 to determine which of the distances to select as the derived distance.

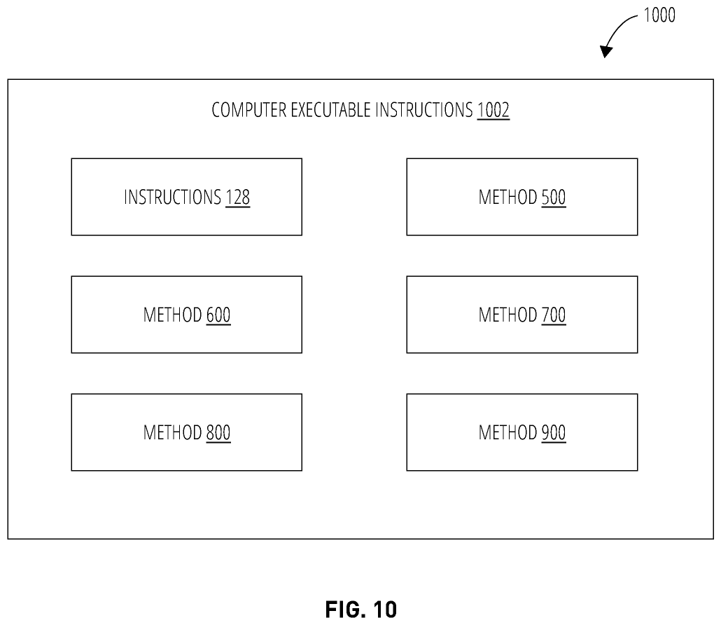

FIG. 10 illustrates computer-readable storage medium 1000. Computer-readable storage medium 1000 may comprise any non-transitory computer-readable storage medium or machine-readable storage medium, such as an optical, magnetic or semiconductor storage medium. In various embodiments, computer-readable storage medium 1000 may comprise an article of manufacture. In some embodiments, computer-readable storage medium 1000 may store computer executable instructions 1002 with which circuitry (e.g., processing unit 112, or the like) of a surgical laser system or console (e.g., surgical laser console 102) can execute. For example, computer executable instructions 1002 can include instructions to implement operations described with respect to instructions 128, method 500, method 600, method 700, method 800, and/or method 900. Examples of computer-readable storage medium 1000 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1002 may include any suitable type of code, such as source code, compiled code, interpreted code, executable code, static code, dynamic code, object-oriented code, visual code, and the like.

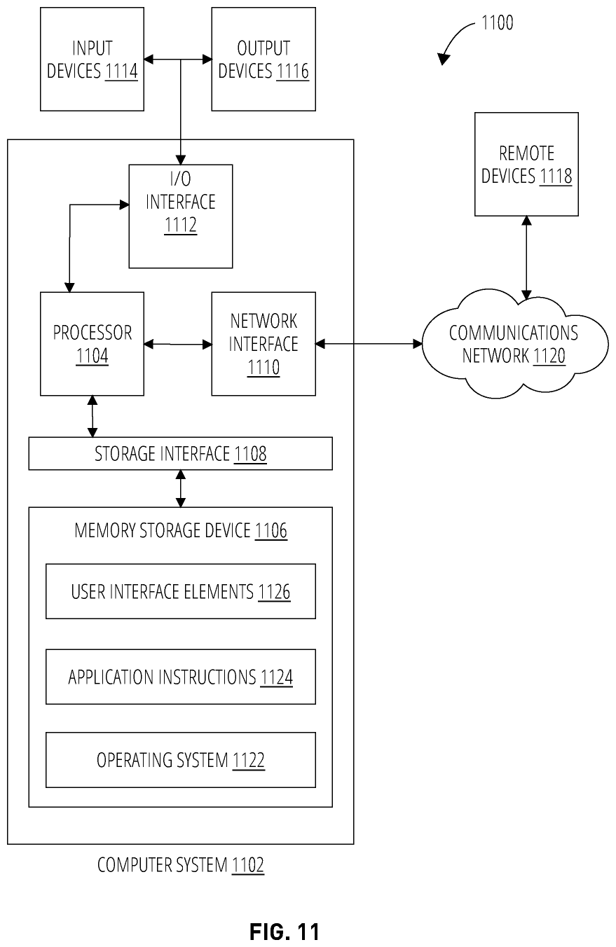

FIG. 11 is a block diagram of a computing environment 1100 including a computer system 1102 for implementing embodiments consistent with the present disclosure. In some embodiments, the computing environment 1100, or portion thereof (e.g., the computer system 1102) may comprise or be comprised in a surgical laser console (e.g., surgical laser console 102, or the like). Accordingly, in various embodiments, computer system 1102 may be used to interpolate a damage to an optical fiber 104 (e.g., proximal end 124 and/or distal end 126) and to control the surgical laser system 100 and/or suggest actions based on interpolation.

The computer system 1102 may include a central processing unit (“CPU” or “processor”) 1104. The processor 1104 may include at least one data processor for executing instructions and/or program components for executing user or system-generated processes. A user may include a person, a person using a device such as those included in this disclosure, or another device. The processor 1104 may include specialized processing units such as integrated system (bus) controllers, memory management control units, floating point units, graphics processing units, neural processing units, digital signal processing units, etc. The processor 1104 may be disposed in communication with input devices 1114 and output devices 1116 via I/O interface 1112. The I/O interface 1112 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 1112, computer system 1102 may communicate with input devices 1114 and output devices 1116. In some embodiments, the processor 1104 may be disposed in communication with a communications network 1120 via a network interface 1110. In various embodiments, the communications network 1120 may be utilized to communicate with a remote memory storage device 1106, such as for accessing look-up tables, performing updates, or utilizing external resources. The network interface 1110 may communicate with the communications network 1120. The network interface 1110 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 1120 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 1120 may include a variety of network devices, including routers, bridges, servers, computing devices, storage devices, etcetera. In some embodiments, the processor 1104 may be disposed in communication with a memory storage device 1106 via a storage interface 1108. The storage interface 1108 may connect to memory storage device 1106 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 1106 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 1106 may store a collection of program or database components, including, without limitation, an operating system 1122, application instructions 1124, and user interface elements 1126. In various embodiments, the operating system 1122 may facilitate resource management and operation of the computer system 1102. Examples of operating systems include, without limitation, APPLE® MACINTOSH® OS X®, UNIX®, UNIX-like system distributions (E.G., BERKELEY SOFTWARE DISTRIBUTION® (BSD), FreeBSD®, NetBSD®, OpenBSD®, etc.), LINUX® DISTRIBUTIONS (E.G., RED HAT® UBUNTU®, KUBUNTU®, etc.), IBM®OS/2®, MICROSOFT® WINDOWS® (XP®, VISTA®/7/8, 10 etc.), APPLE® IOS®, GOOGLE™ ANDROID™, BLACKBERRY® OS, or the like.

The application instructions 1124 may include instructions that when executed by the processor 1104 cause the processor 1104 to perform one or more techniques, steps, procedures, and/or methods described herein, such to irrigate a site and irradiate a site as outlined herein. For example, application instructions 1124, when executed by processor 1104 can cause processor 1104 to perform the method 900.

The user interface elements 1126 may facilitate display, execution, interaction, manipulation, or operation of program components through textual or graphical facilities. For example, user interfaces may provide computer interaction interface elements on a display system operatively connected to the computer system 1102, such as cursors, icons, checkboxes, menus, scrollers, windows, widgets, etcetera. The user interface elements 1126 may be employed by application instructions 1124 and/or operating system 1122 to provide, for example, a user interface with which a user can interact with computer system 1102. As a specific example, the distance 130 derived as outlined herein can be displayed on a display. In some embodiments, the user interface elements 1126 may be integrated with the display (not shown).

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A surgical laser system comprising:
a therapeutic laser source configured to output a therapeutic laser beam;
at least one laser source configured to output an interrogation laser beam;
at least one signal detector configured to measure an intensity of a parasitic reflection at each of the plurality of wavelength components;
a processor; and
a memory comprising instructions, which when executed by the processor cause the surgical laser system to:
generate, via the at least one laser source, the interrogation laser beam,
transmit the interrogation laser beam through an optical fiber coupled to the surgical laser system,
measure, via the at least one signal detector, an intensity of a parasitic reflection, wherein the parasitic reflection comprises at least proximal facet reflections and distal facet reflections,
determine whether the intensity of the parasitic reflection is less than a baseline intensity threshold, and
generate a control signal for the surgical laser system based on a determination that the intensity of the parasitic reflection is less than the baseline intensity threshold.

2. The surgical laser system of claim 1, the instructions when executed by the processor further cause the surgical laser system to:
derive a change in the intensity of the parasitic reflection over a time period; and
determine whether the change in the intensity is greater than an intensity change threshold,
wherein the control signal for the surgical laser system is generated based on a determination that the change in the intensity is greater than the intensity change threshold.

3. The surgical laser system of claim 2, wherein the control signal comprises an indication for the therapeutic laser source and is configured to cause the therapeutic laser source to increase, by a specified amount, a pulse frequency, pulse duration, and/or pulse energy of the therapeutic laser beam generated by the therapeutic laser source.

4. The surgical laser system of claim 2, wherein the control signal comprises an indication for a display and is configured to cause the display to display a suggestion to increase a pulse frequency, pulse duration, and/or pulse energy of the therapeutic laser beam generated by the therapeutic laser source.

5. The surgical laser system of claim 4, the instructions when executed by the processor further cause the surgical laser system to:
generate the therapeutic laser beam via the therapeutic laser source;
illuminate, via the optical fiber, a target with the therapeutic laser beam; and
derive the specified amount of the pulse frequency increase, the pulse duration increase, and/or the pulse energy increase based on the intensity of the parasitic reflection.

6. The surgical laser system of claim 2, wherein the control signal comprises an indication for the therapeutic laser source and is configured to cause the therapeutic laser source to stop generating the therapeutic laser beam.

7. The surgical laser system of claim 2, wherein the control signal comprises an indication for a display and is configured to cause the display to display an indication that an efficiency of the energy delivery via the optical fiber is reduced.

8. The surgical laser system of claim 2, wherein the control signal comprises an indication for a display and is configured to cause the display to display an indication to cleave and/or replace the optical fiber.

9. The surgical laser system of claim 16, wherein the at least one signal detector comprises a first signal detector and a second signal detector and wherein the instructions when executed by the processor further cause the surgical laser system to:
measure an intensity of the proximal facet reflections via the first signal detector; and
measure an intensity of the distal facet reflections via the second signal detector.

10. The surgical laser system of claim 1, the instructions when executed by the processor further cause the surgical laser system to:
measure, via the at least one signal detector, an intensity of a dark current;
determine whether the intensity of the dark current is greater than a dark current baseline threshold; and
wherein the control signal for the surgical laser system is generated based on a determination that the intensity of the dark current is greater than the dark current baseline threshold.

11. The surgical laser system of claim 10, the instructions when executed by the processor further cause the surgical laser system to:
derive normalized intensity based on the measured intensity of the parasitic reflection and the intensity of the dark current; and
determining whether the normalized intensity is less than the baseline intensity threshold to determine whether the intensity of the parasitic reflection is less than the baseline intensity threshold.

12. The surgical laser system of claim 1, the instructions when executed by the processor further cause the surgical laser system to:
illuminate, via the optical fiber, a target with the therapeutic laser beam;
measure, via the at least one signal detector, the intensity of the parasitic reflection and an intensity of a target reflection;
estimate a distance between a distal end of the optical fiber and the target based on the intensity of the parasitic reflection and the intensity of the target reflection.

13. The surgical laser system of claim 1, wherein the interrogation laser beam comprises a plurality of wavelength components and wherein the instructions when executed by the processor further cause the surgical laser system to:
measure, via the at least one signal detector, an intensity at each of the plurality of wavelength components to measure the intensity of the parasitic reflection.

14. At least one non-transitory machine-readable storage device comprising instructions, which when executed by a processor of a surgical laser system cause the surgical laser system to:
generate, via at least one laser source, an interrogation laser beam;
transmit the interrogation laser beam through an optical fiber coupled to the surgical laser system;
measure, via at least one signal detector, an intensity of a parasitic reflection, wherein the parasitic reflection comprises at least proximal facet reflections and distal facet reflections;
determine whether the intensity of the parasitic reflection is less than a baseline intensity threshold; and
generate a control signal for the surgical laser system based on a determination that the intensity of the parasitic reflection is less than the baseline intensity threshold.

15. The at least one non-transitory machine-readable storage device of claim 14, the instructions when executed by the processor further cause the surgical laser system to:
derive a change in the intensity of the parasitic reflection over a time period; and
determine whether the change in the intensity is greater than an intensity change threshold,
wherein the control signal for the surgical laser system is generated based on a determination that the change in the intensity is greater than the intensity change threshold.

16. The at least one non-transitory machine-readable storage device of claim 15, wherein the control signal comprises an indication for the therapeutic laser source and is configured to cause the therapeutic laser source to increase, by a specified amount, a pulse frequency, pulse duration, and/or pulse energy of the therapeutic laser beam generated by the therapeutic laser source.

17. The at least one non-transitory machine-readable storage device of claim 15, wherein the control signal comprises an indication for a display and is configured to cause the display to display a suggestion to increase a pulse frequency, pulse duration, and/or pulse energy of the therapeutic laser beam generated by the therapeutic laser source.

18. The at least one non-transitory machine-readable storage device of claim 17, the instructions when executed by the processor further cause the surgical laser system to:
generate the therapeutic laser beam via the therapeutic laser source;
illuminate, via the optical fiber, a target with the therapeutic laser beam; and
derive the specified amount of the pulse frequency increase, the pulse duration increase, and/or the pulse energy increase based on the intensity of the parasitic reflection.

19. A method for a surgical laser system, comprising:
generating, via a laser source, an interrogation laser beam;
transmitting the interrogation laser beam through an optical fiber coupled to the surgical laser system;
measuring, via at least one signal detector, an intensity of a parasitic reflection, wherein the parasitic reflection comprises at least proximal facet reflections and distal facet reflections;
determining whether the intensity of the parasitic reflection is less than a baseline intensity threshold; and
generating a control signal for the surgical laser system based on a determination that the intensity of the parasitic reflection is less than the baseline intensity threshold.

20. The method of claim 19, further comprising:
deriving a change in the intensity of the parasitic reflection over a time period; and
determining whether the change in the intensity is greater than an intensity change threshold,
wherein generating the control signal for the surgical laser system is further based on a determination that the change in the intensity is greater than the intensity change threshold.
